(19) United States
(12) Patent Application Publication
(10) Pub. No.: US 2006/0235268 A1
(43) Pub. Date: Oct. 19, 2006

(54) Medical device storage apparatus

(75) Inventors: Douglas R. Elsie, Grand Rapids, MI (US); Robert J. Poortvliet, Hudsonville, MI (US); Phillip D. Cammenga, Cedar Springs, MI (US)

Correspondence Address: WARNER NORCROSS & JUDD LLP, 900 FIFTH THIRD CENTER, 111 LYON STREET, N.W., GRAND RAPIDS, MI, 49503-2487, US

(73) Assignee: Innerspace Corporation

(21) Appl. No.: 11/105,212

(22) Filed: Apr. 13, 2005

(51) Int. Cl. A61B 1/00

(52) U.S. Cl. 600/102

(57) ABSTRACT

A storage apparatus for an elongate medical device such as an endoscope. The apparatus includes a mounting bracket and a holder movably coupled to the bracket. The holder is configured to receive a body portion of the medical device, and is moveable between a storage position in which the holder is disposed at a first height, and an accessed position in which the holder is disposed at a second height, less than the first. The apparatus includes an optional tension element that receives an elongate portion of the medical device, which can be draped over the tension element. The tension element engages the elongate portion, and raises it upward, or at least prevents it from descending to the ground, as the holder transitions from the storage position to the accessed position.

### BACKGROUND OF THE INVENTION

The present invention relates to a medical device storage apparatus, and more particularly, to a storage apparatus for storing elongate medical instruments, such as endoscopes.

In recent years, endoscopes have become an integral tool in the medical industry for the identification and treatment of various diseases and conditions. Endoscopes come in a variety of sizes and configurations, but typically include a body having a visual display, and an elongate flexible or rigid tubular portion coupled to the display. The tubular portion is designed for insertion into the body of a patient. Optical instrumentation, for example, lenses, lights and/or fiber optic bundles, is positioned in the end of the elongate portion (opposite the body) to assist in imaging internal organs and tissues of the patient.

Endoscopes and other elongate medical devices usually require specialized storage cabinets. These cabinets usually are designed to hold the devices (a) without bending the elongate portions so that fiber optic bundles or other sensitive components within those portions remain undamaged, and (b) above the ground so that the optical instrumentation is in a safe position. Conventional endoscope storage cabinets usually include a fixed shelf defining a u-shaped recess disposed several feet above the bottom of the cabinet. The endoscope body is supported by the shelf surrounding the recess, and the elongate portion of the scope hangs freely below.

Although conventional endoscope storage cabinets provide storage for endoscopes, they suffer several shortcomings. First, because many endoscopes are relatively long, the shelves on which they are stored must be high above the floor. This makes it difficult for shorter individuals to access the endoscope. Second, conventional storage cabinets provide little protection for the instrumentation in the hanging end of the endoscope. Accordingly, if an inattentive user quickly removes the scope from the cabinet, the hanging end of the scope may drop on the ground and damage the instrumentation. Third, the shelving in endoscope cabinets usually hugs the cabinet wall to reduce the dimension of the cabinet. As a result, individuals with large hands have difficulty accessing and removing the body of the endoscope from the shelf.

### SUMMARY OF THE INVENTION

The aforementioned problems are overcome by a medical device storage apparatus including a mounting bracket and a holder movably coupled to the bracket. The holder is configured to receive a body of the medical device, and is moveable between a storage position in which the holder is disposed at a first height, and an accessed position in which the holder is disposed at a second height, less than the first.

In one embodiment, the apparatus includes an optional tension element that receives an elongate portion of the medical device, which can be draped over the tension element. The tension element engages the elongate portion and raises it upward, or at least prevents it from descending to the ground, as the holder transitions from the storage position to the accessed position.

In another embodiment, the apparatus can include a guide element coupled to at least one of the mounting bracket and the holder. The guide element can optionally be in the form of a housing which covers several of the components of the apparatus. The guide element can include a protrusion and/or a channel which engages the elongate portion of the medical device to align it with the tension element.

The medical device storage apparatus provides a simple and effective mechanism for safely storing and easily accessing elongate medical devices, for example endoscopes. First, because the holder can be moved to a lower height, the apparatus can provide easy access to the stored medical device for users of varying stature. Second, the optional tension element can prevent the hanging end of the medical device from accidentally being dropped to the floor. In turn, this protects costly instrumentation in the end from damage. Third, when moved from the storage position to the accessed position, the holder can move the medical device away from the wall to which the apparatus is secured. This can provide roomy, comfortable access to the device.

These and other objects, advantages and features of the invention will be more readily understood and appreciated by reference to the detailed description of the invention and the drawings.

### BRIEF DESCRIPTION OF THE DRAWINGS

FIG. 1 is a perspective view of a medical device storage apparatus including a housing in a storage position;

FIG. 2 is a perspective view of a medical device storage apparatus including a housing in an accessed position;

FIG. 3 is a perspective view of the medical device storage apparatus without the housing in a storage position;

FIG. 4 is a perspective view of the medical device storage apparatus in transition from a storage position to an accessed position;

FIG. 5 is a perspective view of the medical device storage apparatus in the accessed position; and

FIG. 6. is a section view of a locking feature taken along line 6-6 of FIG. 4.

### DETAILED DESCRIPTION OF THE INVENTION

I. Overview

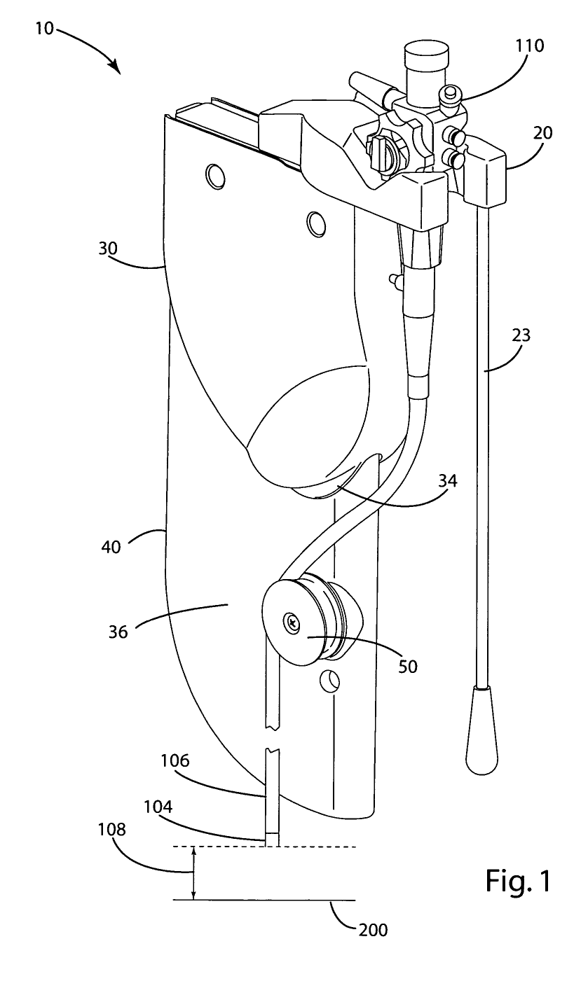

An embodiment of the medical device storage apparatus is shown in FIGS. 1-5 and generally designated 10. The apparatus 10 can include a holder 20 connected to a frame that is disposed under the housing 30, the frame being mounted to a stationary structure, for example, a wall or a cabinet, via a mounting bracket 40. The housing can be configured with an optional guide element 34 in the form of a projection and/or a channel. An optional tension element 50 can also be included in the apparatus.

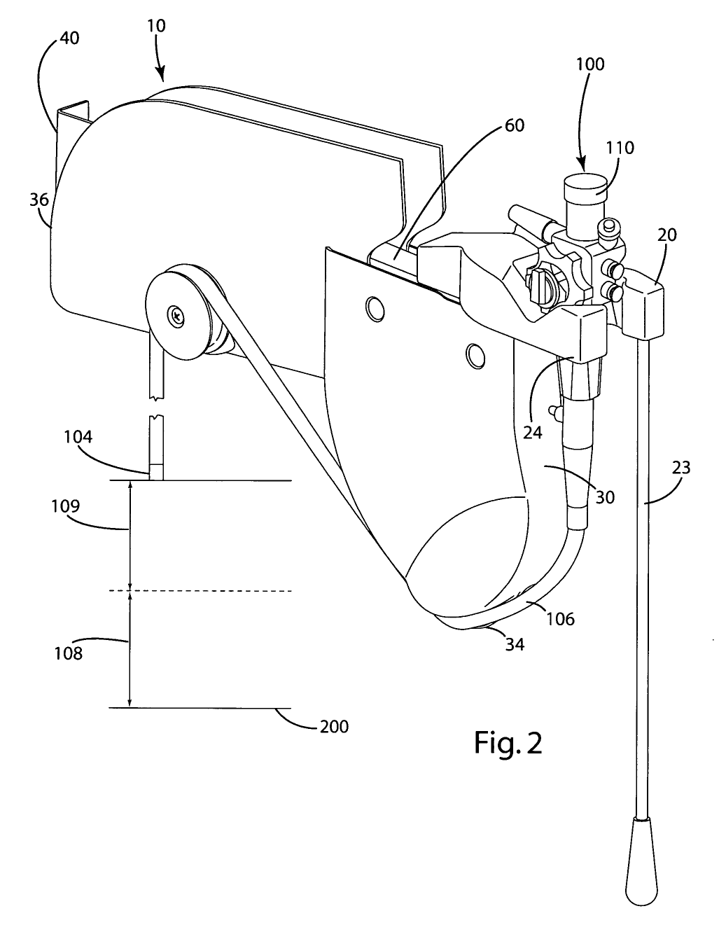

In use, a medical device, for example, an endoscope 100, is stored on the apparatus 10, with the head or body 110 of the device being held by the holder 20. The elongate portion 106 of the endoscope 106 hangs below the holder, and can be draped at least partially over the tension element 50. A distal end 104 of the endoscope, which may include optical or other sensitive instrumentation, hangs below the tension element 50. In the storage position or mode shown in FIG. 1, the distal end is stored a safe distance 108 above the ground. As used herein, ground can refer to a floor, a storage device bottom, or any surface under the storage device. To access the medical device, a user grasps the handle 23 to move the holder 20, and thus the endoscope 100, from the storage mode (FIG. 1) toward an accessed position or mode (FIG. 2). In so doing, the apparatus 10 enables the holder 20 and thus the endoscope 100 to be gradually lowered under a relatively constant force. Simultaneously, the elongate portion 106 of the endoscope rides over the tension element 50, and is aligned with the tension element via guide element 34 when included. In turn, the elongate portion 106 of the endoscope raises so that the endoscope distal end 104 moves away from the ground another distance 109 (or at least moves no closer to the ground). Accordingly, the distal end 104 of the device is protected from collision with the ground as the medical device is accessed.

Although the apparatus 100 is described herein as being an endoscope, the apparatus 10 is well suited for storing a variety of other medical devices that include an elongate portion. Further, the components of the apparatus 10 described herein can be constructed of any material as desired, for example, metal, alloys, synthetic materials and/or combinations of the same.

II. Construction

With reference to FIGS. 1-3, the components apparatus 10 will be described in more detail. As shown in FIG. 1, the apparatus 10 can be outfitted with a primary housing 30 and a secondary housing 36, which cooperate to conceal internal components of the apparatus and provide an aesthetically pleasing appearance. The housing 30 can include a guide element 34, which aligns the endoscope elongate portion 106 with the tension element 50. The guide element 34 can prevent the endoscope from excessive rocking in the holder as the apparatus transitions to an accessed position. The guide element can be in the form of a projection, a channel, or any suitable structure. The housing 30 can be fixedly secured to the secondary bracket 60 using conventional fasteners. The housing 36 can be secured to the mounting bracket 40 so that it rotates as the apparatus 10 transitions from the storage position (FIG. 1) to the accessed position shown (FIG. 2). The housings 30 and 36 can be configured and constructed from materials, such as plastic, to ease sterilization and cleaning when the apparatus 10 is used in hospitals, laboratories and similar environments.

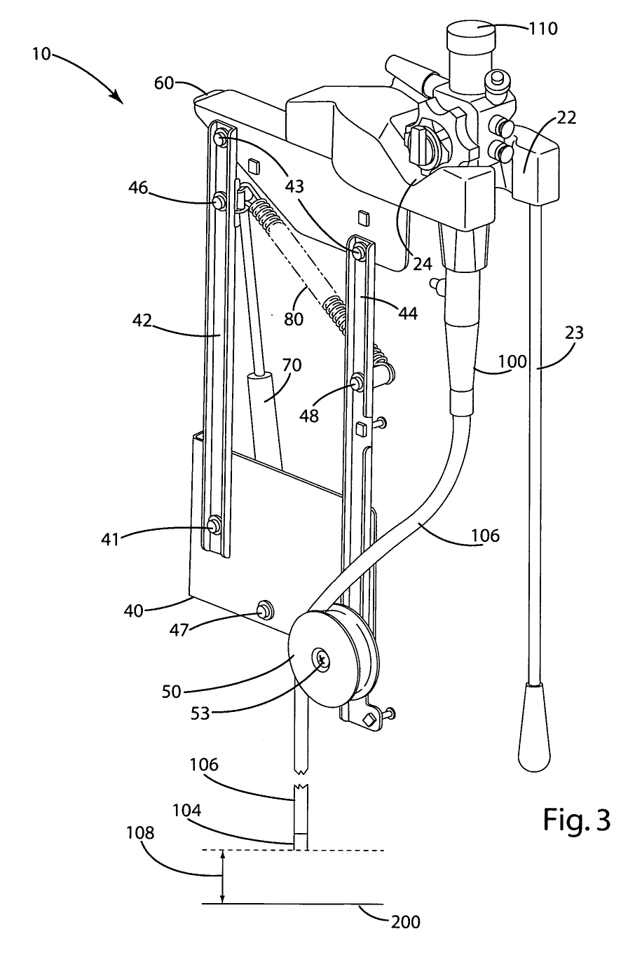

The internal components of the apparatus 10 are shown in FIGS. 3-5. One such component is a mounting bracket 40 which is adapted to mount to a support structure, for example, a cabinet or a wall. The mounting bracket generally remains stationary during operation of the apparatus. The mounting bracket shown includes a forward projecting rectangular plate, and a second plate disposed at an angle to that plate. The second plate can include apertures and/or fasteners which facilitate securing the bracket to a support structure. The bracket 40 can be constructed in any alternative shape or form as desired.

Coupled to and above the mounting bracket 40 is a holder bracket 60 including a holder 20. Although shown as separate components, the holder bracket 60 and holder 20 can be a single, integral unit. The holder 20 can be constructed with an exterior cushion layer adapted to gently contact and/or cradle the endoscope 100. For example, a foam-backed polyvinyl sheet can be wrapped around or over the holder 20, and secured to the holder with an adhesive.

The holder 20 can include a handle 23 which enables a user to grasp the holder and access the endoscope 100. Although shown connected to the holder 20, the handle 23 can be connected to any component of the apparatus 10 above the mounting bracket which enables the user to move the holder 20. The holder 20 can define a recess 22 into which a portion of the endoscope fits. A portion of the holder 20 can further include a stabilization recess 24 to ensure that the head 110 is properly aligned within the recess 22 and/or holder 20.

Optionally, the holder 20 can be configured to receive multiple endoscopes. In such an embodiment, the holder can include multiple recesses 22 and stabilization recesses 24 spaced apart from one another a sufficient distance to accept several endoscopes. The spacing also can be of a specific shape, for example, the recesses can be in a quadrant configuration. Where the holder 20 is configured to hold multiple endoscopes, the apparatus 10 can include multiple tension elements 50 (described below). In such a construction, the mounting bracket and/or housing 36 can be modified to accept the multiple tension elements. For example, the mounting bracket can be extended on opposite sides and include additional brackets to which the additional tension elements mount. Other components of the apparatus 10 can alternatively be modified to support the additional multiple tension elements.

As shown in FIGS. 3-5, the holder bracket 60 is joined with the mounting bracket via the elongate connecting members or elements 42, 44. The elongate members can be of any shape or size as the application requires. For example, although shown as channel-shaped bars, the members can be cylindrical rods or flat strips of rigid material. The elongate members 42, 44 are pivotally secured to the mounting bracket 40 and the holder bracket 60 via fasteners 41, 43. As used herein, fasteners can include any element adapted to join one element to another, including but not limited to bolts, screws, rivets, grommets and other types of fasteners.

The apparatus 10 also includes a biasing element 80 and a compression element 70. The biasing element 80 can be pivotally secured at one end to the first elongate element 42, and at the other end, to the second elongate element 44 with fasteners 46 and 48, respectively. The biasing element 80 can be a coil spring, an elastomeric material, or any body or device that returns to its original shape when released after being distorted. Although shown as being joined with the first elongate member 42 and the second elongate member 44, the biasing element 80 can alternatively be joined with the holder bracket 60 at one end and the second elongate member 44 at the other end, or with the first elongate member 42 at one end and the mounting bracket 40 at the other end, or to the holder bracket 60 at one end and the mounting bracket 40 at the other end, or in any other configuration that slows the return of the apparatus 10 to the storage position.

The compression element 70 can be pivotally secured at one end to the first elongate element 42 with fastener 46, and at the other end, to the mounting bracket 40 with fastener 47. Although shown as a gas cylinder spring, the compression element 70 can be any body or device that re-expands to its original shape or dimension when released after being distorted. Furthermore, although shown as pivotally joined with the first elongate member 42 and the mounting bracket 40, the compression element 70 can be joined at one end with the holder bracket 60 and the mounting bracket 40 at the other end, or in any other configuration that urges the apparatus 10 to return to the storage position.

A tension element 50 can be mounted to the mounting bracket 40. As shown, the tension element is in the form of a reel, which is rotatably mounted to the bracket 40, including opposing flanges that form a channel within which the elongate portion 106 of the endoscope can rest. The tension element can alternatively be joined to either of the elongate connecting members 42 or 44, or even to the holding bracket 60, or the holder 20, or either of the housings 30, 36. In one such alternative embodiment, a threaded insert (not shown) can be associated with the housing 36, for example by heat inserting the insert into the housing. A threaded fastener can be coupled to the insert. This fastener can be an axle on which the reel is mounted. Although shown as a rotatable reel, the tension element can be a fixed non-rotatable reel or spool, or even a member projecting from the remainder of the apparatus 10 and adapted so that the endoscope elongate portion 106 can be draped over that element.

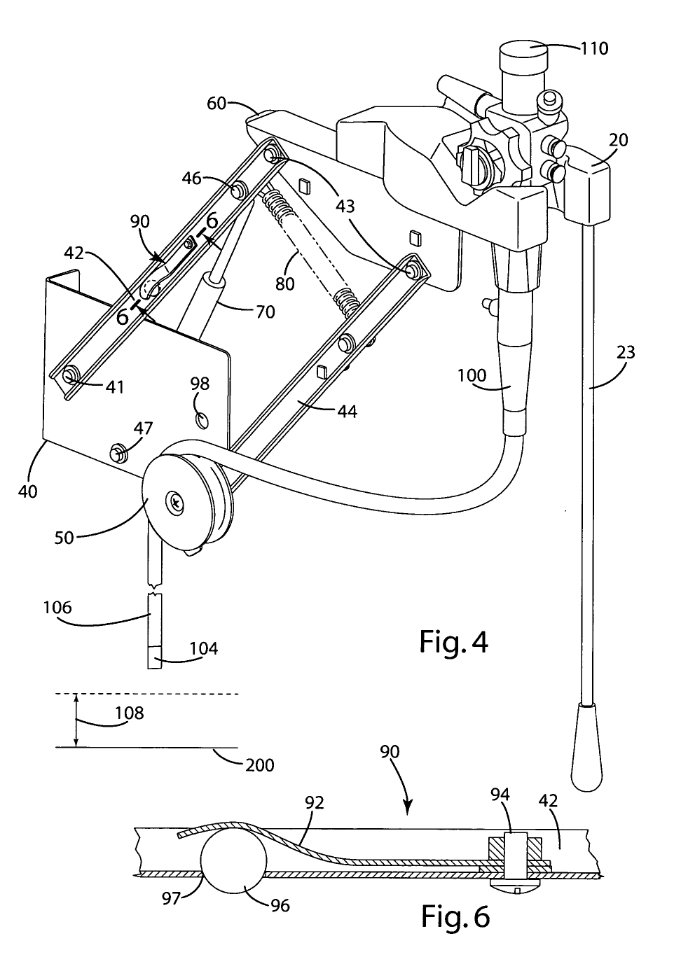

Another optional feature that can be included with the apparatus is a locking feature 90, shown in FIGS. 4 and 6. The locking feature 90 includes a leaf spring 92 secured to a component of the apparatus 10, for example the elongate member 42, via a fastener 94. The leaf spring presses a bearing 96 so that it registers in a hole 97 defined by the member 42 (or whatever component to which the locking feature is coupled). A portion of the bearing 96 projects through the hole 97. Another component of the apparatus, for example the mounting bracket 40, defines another hole 98. The locking feature 10 operates such that when the apparatus is transitioned to the accessed position, the bearing partially retracts from the hole 97, rides over a portion of the mounting bracket 40, until seating again in the hole 97, and at least partially in the hole 98 defined by the mounting bracket 40. Accordingly, the feature temporarily locks the apparatus in the accessed position.

III. Method of Manufacture and Use

Methods for manufacturing and using the medical device storage apparatus of the present invention will now be described with reference to FIGS. 1-5. To begin, the components of the apparatus 10 are constructed using conventional manufacturing techniques and materials. In assembly, the holder bracket 60 is secured to the mounting bracket 40 via the elongate connecting members 42, 44. This is accomplished by securing the fasteners 43 and 41 to these components. The holder 20 can be secured to the holder bracket 60 with one or more fasteners (not shown) and held in a fixed position relative to that bracket. The compression element 70 and biasing element 80 are secured to the respective elongate connecting members 42, 44 and mounting bracket 40 as shown. The tension element 50 can be fastened to the mounting bracket 40 with the same fastener 53 used to connect the elongate element 44 to the mounting bracket 40.

In use, an endoscope 100 is positioned on the apparatus 10 as shown in FIG. 3 such that the body 110 of the endoscope rests in the recess 22 defined by the holder. The body can further rest in the optional stabilization recess 24 to ensure that the body 110 is properly aligned within the recess 22. The elongate portion 106 of the endoscope, which is typically flexible and several feet long, hangs below the holder. When a tension element 50 is included with the apparatus 10, the endoscope elongate portion 106 can be draped over the tension element 50 as shown in FIGS. 3-5. In one embodiment, the tension element 50 is positioned relative to the remainder of the components of the apparatus so that the distal end 104 of the endoscope is maintained a safe distance 108 above the ground 200.

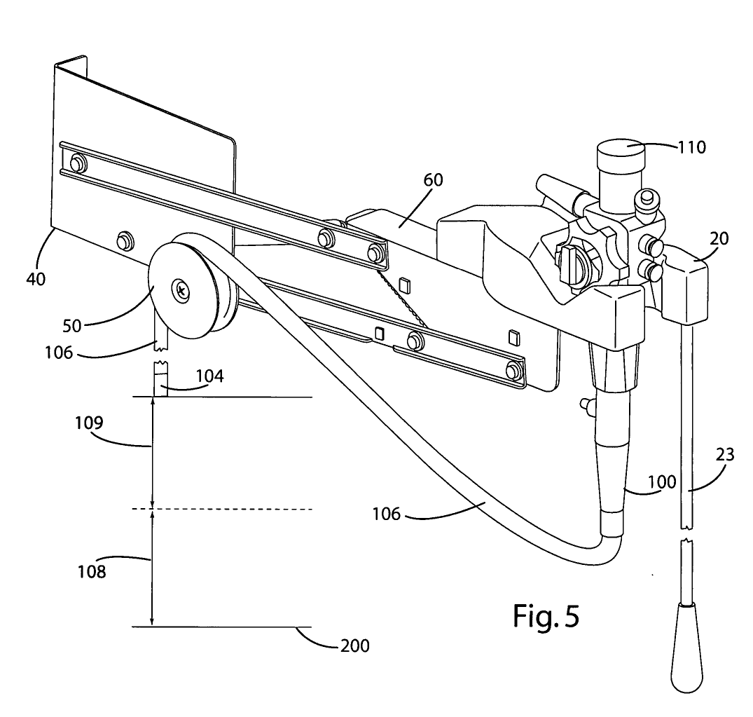

To move the storage apparatus 10 from the storage position shown in FIG. 3 to the accessed position shown in FIG. 5, a user grasps the handle 23 and begins to pull generally downward on the handle. The biasing element 80 assists the user in so moving the holder 20 by counteracting the compression element 70, which urges the holder 20 to the storage position. In one embodiment, the biasing element 80 and compression element 70 interact so that transitioning from the storage position to or toward the accessed position can be performed by a user applying to the handle 23 a relatively constant force, for example, about two to three pounds.

As shown in FIGS. 3-5, as the holder transitions to a lower height, the tension element 50, being in a fixed location relative to the ground 200, assists in raising the distal end 104 of the endoscope an additional distance 109 away from the ground. Accordingly, when a user removes the endoscope 100 from the holder, the distal end 104 is raised that sufficient additional distance 109 above the ground 200 to minimize the likelihood that the end 104 will collide with the ground when removed completely from the apparatus. This can further protect any instrumentation in the distal end 104 of the endoscope.

Where included, the locking feature 90 (FIGS. 4 and 6) can temporarily lock the apparatus in the accessed position. To disengage the locking feature, a user need only push the holder toward the stored position. This will urge the bearing 96 against the resilient leaf spring 92, and accordingly disengage the bearing 96 from the mounting bracket hole 98.

After the endoscope 100 is removed from the apparatus and the handle 23 is released (and the optional locking feature 90 is disengaged), the stored energy in the compression element 80 returns the holder toward and/or to the stored position. The biasing element 70 concurrently counteracts the compression element to ensure that the holder 20 is not catapulted at an unmanageable speed to the storage position.

Further, if the endoscope is placed on the holder 20 when the apparatus is in the accessed position, the compression element 80 and biasing element 70 cooperate to move the endoscope in a relatively slow motion toward the stored position. Additionally, during such movement, the distal end 104 of the endoscope is lowered slowly and securely toward the ground until it rests a safe distance away 108 from the ground.

The above descriptions are those of the preferred embodiments of the invention. Various alterations and changes can be made without departing from the spirit and broader aspects of the invention as defined in the appended claims, which are to be interpreted in accordance with the principles of patent law including the doctrine of equivalents. Any references to claim elements in the singular, for example, using the articles “a,” “an,” “the,” or “said,” is not to be construed as limiting the element to the singular.

The embodiments of the invention in which an exclusive property or privilege is claimed are defined as follows:

1. A storage apparatus for an elongate medical device comprising:
a mounting bracket adapted to mount the apparatus to a structure;
a holder bracket including a holder, the holder defining a recess configured to receive a portion of the medical device;
an elongate first connecting element including a first end and a second end, the first end pivotally joined with the holder bracket, the second end pivotally joined with the mounting bracket;
an elongate second connecting element including a third end and a fourth end, the third end pivotally joined with the holder bracket, the fourth end pivotally joined with the mounting bracket;
a compression element joined with the first connecting element and the mounting bracket;
a biasing element joined with the first connecting element and the second connecting element; and
a reel rotatably joined with the mounting bracket, whereby a medical device including a head and an elongate portion is held in the storage unit with the head resting at least partially on the holder, the elongate portion hanging below the holder and at least partially draped over the reel.

2. The storage apparatus of claim 1 wherein the compression element and the biasing element are connected to the first connecting element at a common pivot point.

3. The storage apparatus of claim 1 wherein the holder bracket is moveable from a storage position to an accessed position.

4. The storage apparatus of claim 3 wherein the reel engages the elongate portion of the medical device when the holder bracket moves from the storage position to the accessed position to lift at least a portion of the elongate portion upward.

5. The storage apparatus of claim 4 wherein the holder moves forward relative to the mounting bracket when the holder bracket moves from the storage position to the accessed position.

6. The storage apparatus of claim 1 comprising a housing having a guide element which aligns the elongate portion of the medical device with the reel.

7. A storage apparatus for an elongate medical device comprising:
a mounting bracket adapted to mount the apparatus to a structure;
a holder bracket including a holder, the holder defining a recess configured to receive a body portion of the medical device, the holder bracket coupled to the mounting bracket and moveable between a storage position in which the holder is disposed at a first height, and an accessed position, in which the holder is disposed at a second height being less than the first height; and
a tension element disposed below the holder and adapted to receive an elongate portion of the medical device draped over the tension element, the tension element engaging the elongate portion of the medical device to raise at least a portion of the elongate portion as the holder bracket transitions from the storage position to the accessed position.

8. The storage apparatus of claim 7 wherein the tension element is a reel rotatably mounted to the mounting bracket.

9. The storage apparatus of claim 7 comprising a guide element coupled to at least one of the mounting bracket and the holder bracket which aligns the elongate portion of the medical device with the tension element.

10. The storage apparatus of claim 7 comprising a compression element joined with the mounting bracket, the compression element adapted to urge the holder bracket to the storage position.

11. The storage apparatus of claim 7 comprising a first elongate element and a second elongate element, each pivotally joined with both the mounting bracket and the holder bracket.

12. The storage apparatus of claim 11 comprising a coil spring joined with the first and second elongate members, wherein the coil spring assists in moving the holder bracket from the storage position toward the accessed position.

13. The storage apparatus of claim 12 comprising a gas cylinder spring joined with the mounting bracket, the gas cylinder spring adapted to urge the holder bracket to the storage position, the coil spring countering the gas cylinder spring when the gas cylinder spring urges the holder bracket to the storage position.

14. A storage apparatus for an elongate medical device comprising:
a mounting bracket adapted to mount the apparatus to a structure;
a holder configured to receive a body portion of the medical device, the holder joined with the mounting bracket, the holder moveable between a storage position in which the holder is disposed at a first height, and an accessed position, in which the holder is disposed at a second height, less than the first; and
a tension element disposed below the holder and adapted to receive an elongate portion of the medical device draped over the tension element, the tension element engaging the elongate portion of the medical device to prevent the elongate portion from dropping to the ground as the holder bracket transitions from the storage position to the accessed position.

15. The storage apparatus of claim 14 comprising at least one elongate element that joins the holder and the mounting bracket, the elongate element pivotally mounted to the mounting bracket.

16. The storage apparatus of claim 15 comprising a gas cylinder spring joined with the elongate element and the mounting bracket, the gas cylinder spring adapted to urge the holder to the storage position.

17. The storage apparatus of claim 16 comprising a coil spring joined with the elongate element, the coil spring urging the holder from the storage position to the accessed position.

18. The storage apparatus of claim 14 wherein the tension element is a reel rotatably mounted to the mounting bracket.

19. The storage apparatus of claim 14 comprising a locking feature that engages at least one of the holder and the mounting bracket to temporarily lock the holder in the accessed position.

20. The storage apparatus of claim 14 comprising a guide element coupled to at least one of the mounting bracket and the holder which aligns the elongate portion of the medical device with the tension element.
